(19) United States
(12) Patent Application Publication
(10) Pub. No.: US 2022/0265371 A1
(43) Pub. Date: Aug. 25, 2022

(54) Generating Guidance Path Overlays on Real-Time Surgical Images

(71) Applicant: Asensus Surgical US, Inc., Durham, NC (US)

(72) Inventors: Kevin Andrew Hufford, Durham, NC (US); Caleb T. Osborne, Durham, NC (US); Arun Mohan, Durham, NC (US); Lior Alpert, Durham, NC (US); Carmel Magan, Karmi'el, NC (US)

(73) Assignee: Asensus Surgical US, Inc., Durham, NC (US)

(21) Appl. No.: 17/679,021

(22) Filed: Feb. 23, 2022

Related U.S. Application Data
(60) Provisional application No. 63/152,833, filed on Feb. 23, 2021.

(51) Int. Cl. A61B 34/00; G06T 7/73; G06F 3/04845; G06T 11/20; A61F 5/00; A61B 1/00; A61B 1/04; A61B 17/072

(52) U.S. Cl. CPC A61B 34/25; G06T 7/73; G06F 3/04845; G06T 11/203; A61F 5/0086; A61B 1/0005; A61B 1/04; A61B 17/072; G06T 2207/10068; G06T 2207/30092; G06T 2200/24; G06T 2210/41; A61B 2034/107

(57) ABSTRACT

In a system and method for determining guide points or a guide path for display on an endoscopic display, image data corresponding to a surgical treatment site is captured using a camera. Using the image data, the positions of one or more reference points within the surgical environment, are determined. Based on the positions of the reference points, the positions of guide points spaced from the reference point are estimated or determined, in some cases using predetermined offsets. The guide points or guide path is displayed as an overlay of the image data on an image display. In an embodiment using the system for a sleeve gastrectomy procedure, the reference points are input by a user or determined by the system with reference to a bougie that has been positioned within a stomach at the operative site, and the guide path is used as a guide for stapling and resection to form the sleeve.

This application claims the benefit of U.S. Provisional Application No. 63/152,833, filed Feb. 23, 2021

### BACKGROUND

Sleeve gastrectomy, or vertical sleeve gastrectomy, is a surgical procedure in which a portion of the stomach is removed, reducing the volume of the stomach. The resulting stomach typically has an elongate tubular shape.

Referring to FIG. 1A, a typical sleeve gastrectomy involves use of an elongate stomach bougie 200 that aids in defining the stomach sleeve or pouch to be formed, and a surgical stapler 202 to be used to resect and fasten the stomach tissue to form the sleeve. In use, a bougie of a size selected by the surgeon is positioned to extend through the stomach from the esophagus to the pylorus. The surgeon typically feels for the bougie with an instrument positioned at the stomach, such as the stapler that will be used to form the sleeve, prior to beginning the staple line. The surgeon forms the sleeve by maneuvering the stapler, using the bougie as a guide. FIG. 1B shows the stomach after the stapler (not shown in FIG. 1B) has been fired twice. The stapler is repositioned after each staple reload is fired, until the sleeve is completed (FIG. 1C).

The size of the finished sleeve is dictated by how close the surgeon gets the stapler to the bougie, the size of the bougie and whether or not the surgeon over-sews the staple line. The distance between the stapler and the bougie is defined only by the surgeon's estimation. In other surgical procedures, the surgeon may wish to stay at least a certain distance away from a defined anatomical structure (e.g. a critical blood vessel) or another surgical instrument, or to be no further than a certain distance from an anatomical structure or another surgical instrument.

This application describes systems and methods that generate procedure guidance using real-time measurements or other input from the surgical environment to aid a user in defining pathways for stapling, cutting, or other surgical steps, and/or in defining key regions such as keep-out zones or keep-within zones. These concepts may be used with or incorporated into surgical robotic systems, such as the Senhance System marketed by Asensus Surgical, Inc or alternative systems, or they may be used in manually performed surgical procedures.

### BRIEF DESCRIPTION OF THE DRAWINGS

FIGS. 1A-1C show a sequence of drawings that schematically depict a surgical method in which a stomach pouch is created along a bougie positioned to extend from the esophagus to the pylorus;

FIG. 2A is a block diagram schematically illustrating a system according to the disclosed embodiments.

FIG. 2B is a functional diagram setting forth general steps carried out by the system depicted in FIG. 2A.

FIGS. 3A through 8 show an example of a graphical user interface (GUI) displaying an image of a surgical site during use of the system in a staple line planning mode, in which:

FIG. 3A shows the displayed image where the stomach is lying flat and the bougie is being passed into the stomach from the esophagus.

FIG. 3B is similar to FIG. 3A and shows overlays marking the path or edge of the bougie and an offset line generated with reference to the path.

FIG. 4 is similar to FIG. 3B, but further displays lines extending between the reference line and the offset line, with dimensional information displayed representing the length of the extending lines.

FIG. 5 is similar to FIG. 4, but further shows an example of an informational overlay informing the user that the system is in a staple line planner mode and providing information as to what the lines and markings displayed as overlays represent.

FIG. 6 shows the image display during staple line planning in accordance with an alternative embodiment performed with reference to an edge of the stomach, which has been marked with an overlay on the image display.

FIG. 7 shows the image display in accordance with an alternative embodiment in which markings intraoperatively placed on the stomach by the surgeon are recognized by the system and marked on the image display using overlays of icons;

FIG. 8 is similar to FIG. 7, but further shows an overlay of a reference line extending between the icons, and an overlay of a suggested staple line positioned between the reference line and the overlay following the stomach edge.

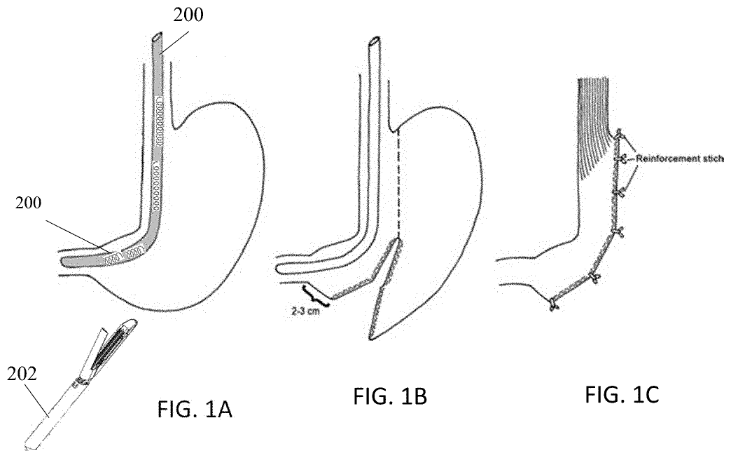

### DETAILED DESCRIPTION

This application describes systems and methods that display visual guides as overlays on a display of a real time image of a surgical site, so that the user may reference the visual guides when guiding a manual or laparoscopic instrument to treat tissue (e.g. staple, cut, suture etc.). The locations for the visual guides are determined by the system with reference to reference points or lines. In some embodiments, the reference points or lines are input by a user observing the real time image display. In other embodiments, the reference points or lines are additionally or alternatively determined by the system by analyzing real time images of the surgical site and using computer vision techniques to recognize features, landmarks, or changes in the surgical site, as will be described in greater detail below. In some cases, the visual guides are separated by the reference points or lines based on predetermined, user-input, or user-selected offset distances.

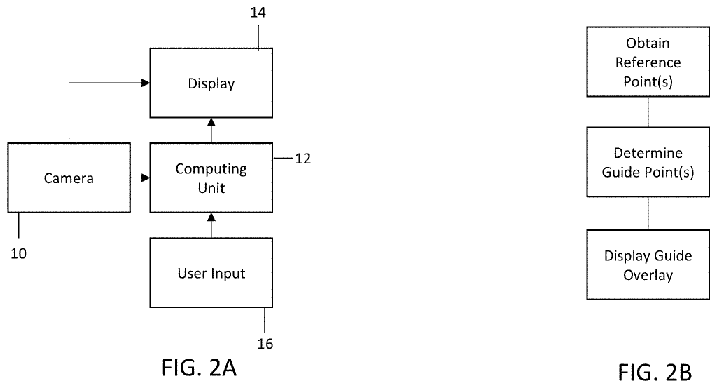

Referring to FIG. 2A, an exemplary system preferably includes a camera 10, one or more processors 12 receiving the images/video from the camera, and a display 14. The camera may be a 2D camera, but it is preferably a 3D camera, such as one comprising a pair of cameras (stereo rig), or structured light-based camera (such as Intel RealSense™ camera), or a 2D camera using other software or hardware features that allow depth information to be determined or derived.

The processor(s) includes at least one memory storing instructions executable by the processor(s) to (i) obtain one or more reference points and determine the (preferably 3D) positions of the one or more reference points within the surgical environment, (ii) based on the positions of the reference points and defined offsets, estimate or determine (preferably 3D) positions of guide points, which are points in the surgical site that are spaced from the reference point(s) by a distance equivalent to the amount of the offsets and (iii) generate output communicating the positions of the guide point(s) to the user. These steps are depicted in FIG. 2B. The output is preferably in the form of graphical overlays on the image display displaying guide data (e.g. as points or lines) (as described in connection with the drawings), and/or in other forms such as haptic output (where the system is used in conjunction with a surgical robotic system), or auditory output.

In many embodiments, user input is used for one or more purposes. For example, a user may use input device(s) to input reference points to the system, to give input to the system that the system then uses to identify reference points, and/or to specify, select or adjust offsets. The system may therefore include one or more input devices 16 for these purposes. When included, a variety of different types of user input devices may be used alone or in combination. Examples include, but are not limited to the following devices and methods, and examples of how they might be used to identify measurement points when the system is in a measurement point input mode of operation:
Eye tracking devices. The system determines the location at which the user is looking on the display 14 and receives that location as input instructing the system to set that location as a reference point. In a specific implementation, when in a mode of operation in which the system is operating to receive a user-specified reference point or line, the system displays a cursor on the display at the location being viewed by the user, and moves the cursor as the user's gaze moves relative to the display. In this and the subsequently described examples, confirmatory input (discussed below) can be input to the system confirming the user's selection of a reference point, or confirmation that a reference line drawn by the user using gaze input should be input as reference input.
Head tracking devices or mouse-type devices. When the system is in a reference point input mode of operation, the system displays a cursor on the display and moves the cursor in response to movement of the head-worn head tracking device or movement of the mouse-type of device.
Touch screen displays, which display the real time image captured by the camera. The user may input a desired reference point by touching the corresponding point on the displayed image, or draw a reference path or line on the touchscreen.
If the system is used in conjunction with a surgical robotic system, movement of an input handle that is also used to direct movement of a component of a surgical robotic system. Input handles may be used with the operative connection between the input handle and the robotic component temporarily suspended or clutched. Thus the input handle is moved to move a cursor displayed on the display to a desired reference point. Confirmatory input is used to confirm a current cursor position as a selected reference point.
Alternative, the cursor may be dragged to draw a reference line that is used as a collect of reference points.
Movement of another component on the input handle for a robotic surgical system, such as a joystick, touchpad, trackpad, etc.; Manual or robotic manipulation of a surgical instrument (with the robotic manipulation performed based on using input from an input handle, eye tracker, or other suitable input device) within the surgical field. For example, the instrument may have a tip or other part (e.g. a pivot of a jaw member, rivet, marking) that is tracked using image processing methods when the system is in an instrument-as-input mode, so that it may function as a mouse, pointer and/or stylus when moved in the imaging field, etc. The tracked part may be recognized by the system or identified to the system by the user. Alternatively or additionally, a graphical marking can be displayed on the display over or offset from the instrument. These icons are moved by the user through movement of the surgical instrument (manually or by a robotic manipulator that moves the instrument in response to user input). Where robotically manipulated surgical instruments are used to identify reference points to the system, the positions of the reference points may be calculated using only the image data captured using the camera, and/or using information derived from the kinematic data from the robotic manipulators on which the instruments are mounted.
The system may be configured or placed in a mode so that the reference points are recognized on the image using computer vision. Such points might include points on surgical devices or instruments (e.g. tips or other structural features, or markings) recognized by the system, edges or other features of tissue structures or tissue characteristics, etc., physical markings or markers placed on the tissue itself (e.g. marks drawn on the surface of the stomach using a felt tip pen, one or more stitches placed in the stomach surface using suture material). U.S. application Ser. No. 17/035,534, entitled “Method and System for Providing Real Time Surgical Site Measurements” (TRX-28600R) describes techniques that may be used for identifying structures or characteristics.
Voice input devices, switches, etc.

Input devices of the types listed are often used in combination with a second, confirmatory, form of input device allowing the user to enter or confirm the selection of a reference point, or confirmation that a reference line drawn by the user using an input device should be input as reference input. If a user input for a robotic system is used, confirmatory input devices might include a switch, button, touchpad, trackpad on the user input used to give input for robotic control of the surgical instruments. Other confirmatory inputs for use in robotic or non-robotic contexts include voice input devices, icons the user touches on a touch screen, foot pedal input, keyboard input, etc.

Reference Point(s)/Path

The term “reference points” is used in this application to mean one or more discrete points, or collections of points forming paths or lines.

The reference point(s), lines, paths etc. may be input to the system by a user, or determined by the system with or without input from the user. Various non-limiting examples are given in this section.

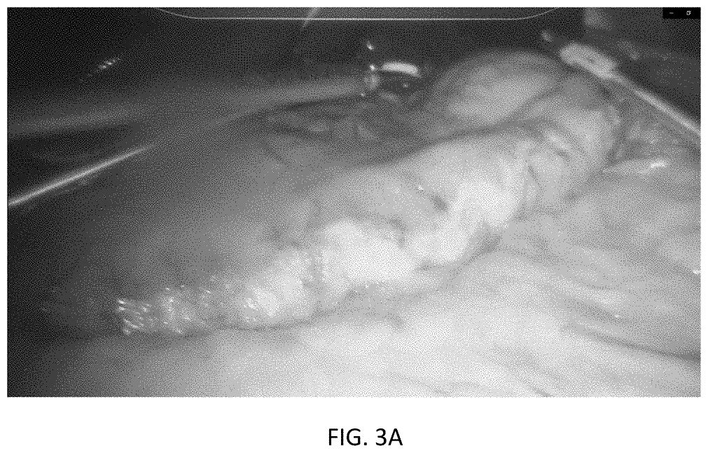

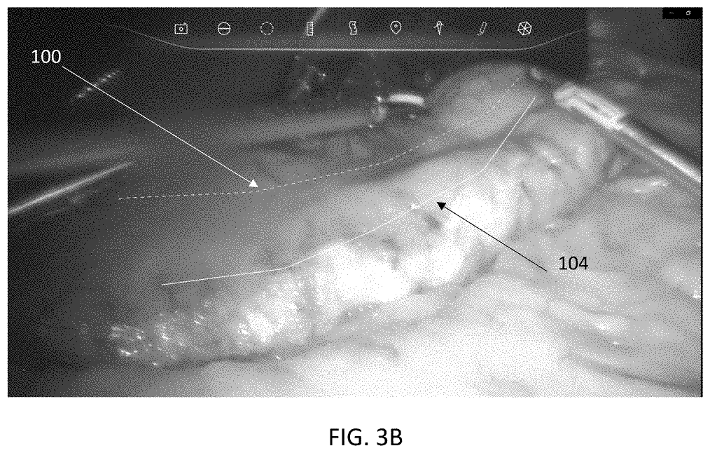

According to a first example, reference points are input by a user viewing an image display showing the image captured by the endoscopic camera. In this example, the user “draws” a reference path, which the system then displays as a graphical overlay on the image display, using a user input device. See, for example, FIG. 3B, in which, during a sleeve gastrectomy procedure, the user has followed the shape of the bougie using a user input device to draw reference path 100. In a modified example, the user inputs a discrete number of reference points along a path (e.g. the endpoints of a desired path with or without intermediate points between the endpoints) and the system determines the path between the reference points. The determined path may be one that smoothly connects the input points, or it may be comprised of straight line segments between adjacent pairs of reference points, and/or the geodesic path between the reference points, the latter being determined by the processor using 3D image data obtained or generated using the camera image and taking into account the variations in depth of the surface features (e.g. the tissue surface) along the path between pairs of the reference points. Note that when the geodesic path is determined, the reference points are preferably attached to the locations at the appropriate depth of the tissue or other structure within the body cavity at which the user-placed reference points have been positioned (as determined using the system, rather than floating above the tissue at some point in space). These concepts are discussed in greater detail in co-pending U.S. application Ser. No. 17/099,761, filed Nov. 16, 2020 (“METHOD AND SYSTEM FOR PROVIDING SURGICAL SITE MEASUREMENT”) which is incorporated herein by reference. In other embodiments, the shape of the path may be determined by the processor based on other input, such as the shape of the external edge of the stomach, as discussed in greater detail below.

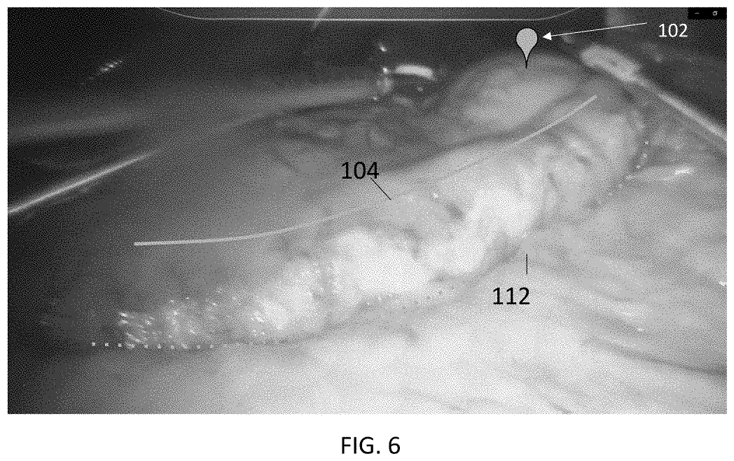

In a second example, all or some of the reference points that are ultimately used to define the path are determined by the system. For example, the system might recognize the locations of anatomical landmarks. In a specific embodiment, the system recognizes one or more portions of the bougie beneath the stomach wall using computer vision. In this embodiment, the system may recognize the shape of the stomach surface as having been shaped by the bougie, and/or it may recognize changes in the shape of the stomach surface resulting from placement or movement of the bougie, and/or it may recognize movement of the stomach surface during advancement or maneuvering of the bougie. The processor might generate and cause the display of an icon 102 overlay on the displayed endoscopic image, and the system might prompt the user for input confirming that the location of the icon 102 is one desired as a reference point. See FIG. 6. Computer vision might also be used to recognize physical markers positioned on the stomach, such as markings on the tissue made using a pen or dye, or stitches formed in the tissue using suture. Recognized points may be supplemented by additional reference points input by the user. Once reference points are identified, the processor creates a path connecting the reference points, as described in the first example. Related concepts which may combined with those discussed here are described in commonly owned U.S. application Ser. No. 16/733,147, filed Jan. 2, 2020 (“Guidance of Robotically Controlled Surgical Instruments Along Paths Defined with Reference to Auxilliary Instruments”) which is incorporated herein by reference.

Once the reference path is determined, it is preferably displayed as an overlay on the endoscopic image display.

Guide Points/Path

Once the reference path is determined, the processor determines a guide path that is offset from the reference path. The guide path may be referenced by a surgeon for a variety of purposes. In the sleeve gastrectomy example, the guide path is a path the surgeon references when forming the staple line. In other contexts, the guide path is a path marking a boundary the surgeon does not want to cross with surgical instruments (defining a keep-out zone or a stay-within zone).

The distance by which the guide path is spaced from the reference path (the “offset”) may be set in a number of different ways. A user may give input to the system setting the desired offset(s), preoperatively or intraoperatively.

While the guide path might run parallel to the reference path (i.e. has a constant offset), it may be preferable to offset the guide path from the reference path by different amounts in different regions. For example in a sleeve gastrectomy, the offset distance may vary along the path, such as at the entrance and exit of the stomach.

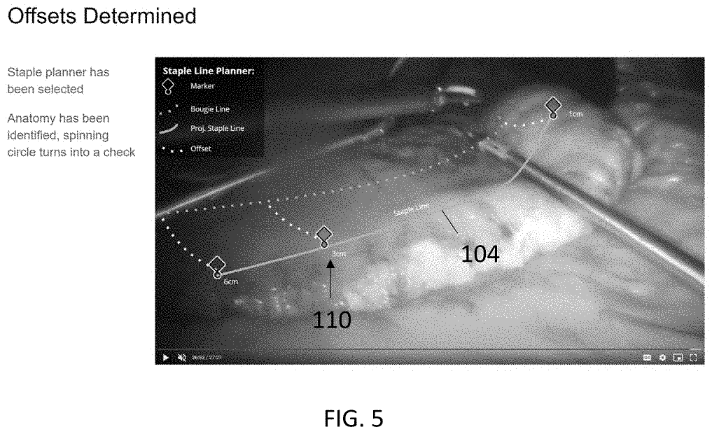

In some embodiments, the guide path is generated using predetermined or pre-set offsets, and then the user can give input instructing the system to modify the offsets. For example, in the FIG. 5 example, offsets of 6 cm and 1 cm are used at different ends of the stomach, and an intermediate offset of 3 cm is used. The system may be configured to allow the user to adjust any one, or all, of these offsets. For example, the system may be set up to allow the user to adjust one of the displayed offsets by dragging an edge of the overlay marking the guide path, or by dragging a marker that is positioned along the guide path overlay. The system might also be set up to allow a user to cause movement of the entire guide path overlay towards or away from the reference line while maintaining its shape, by dragging the guide path overlay or using alternate input. Where offset distances are displayed on the image display as in FIG. 5, moving all or a portion of the guide path overlay may result in re-calculation of the offset measurements and display of the updated measurements. The distance measured may be the straight line “ruler distance” between the measurement points on the reference path and guide path, and/or the geodesic distance between the points, which takes into account the variations in depth of the surface features (e.g. the tissue surface) along the line between the two points, as discussed above. Note that these measurement points are preferably attached to the locations at the appropriate depth of the tissue or other structure within the body cavity at which a measurement is being take, (as determined using the system, rather than floating above the tissue at some point in space). Relevant measurement concepts are discussed in greater detail in co-pending U.S. application Ser. No. 17/099,761, filed Nov. 16, 2020 (“METHOD AND SYSTEM FOR PROVIDING SURGICAL SITE MEASUREMENT”) which is incorporated herein by reference.

The processor may additionally be programmed to take other parameters into consideration when determining the guide path. For example, the external edge of the stomach may be recognized in the camera image using computer vision and used by the system to determine an initial shape for the guide path (e.g. a guide path might be determined that parallels the edge). In this example, the position of the bougie (as input by the user or determined by the system) or other placed reference points may also be used to refine this shape and to fine tune the offsets along the guide path.

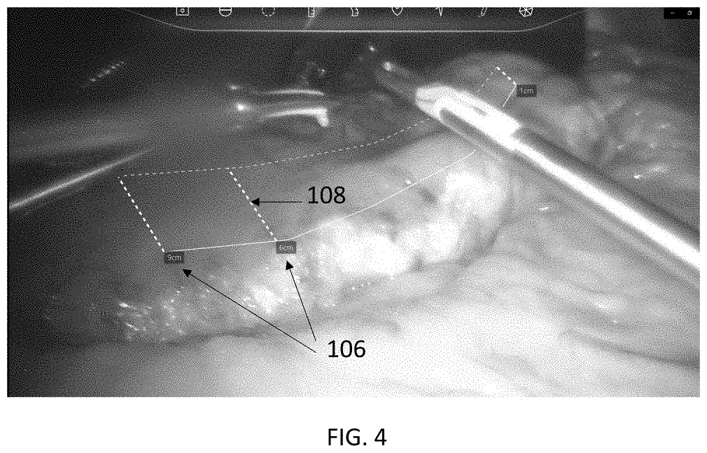

Some specific embodiments will next be described with respect to the drawings. FIG. 3A shows the endoscopic image, in which a stomach is seen lying flat as a bougie is being introduced into it via the esophagus. Next, the reference path is drawn or determined, using any of the methods described above, and an overlay of the reference path 100 is displayed as an overlay on the endoscopic display. See FIG. 3B. A guide path is determined using any of the methods described above, and an overlay of the guide path 104 is displayed. As shown in FIGS. 4, offset distance measurements 106 for various points along the guide path may be shown. The paths 108 along which those measurements are taken may also be shown.

The user may give input to the system identifying points for which the display off an offset distance is sought, and/or the system may automatically generate offset distance measurements at predetermined points along the guide path. If desired, the offsets may be increased or decreased, such as by dragging the markers 110 shown in FIG. 5 marking points on the guide path at which the offset measurements are taken, dragging the guide path overlay 104, or in other ways including those described above.

In a second embodiment shown in FIG. 6, the system recognizes the presence of the bougie in the stomach, using techniques such as those described above. A waypoint or landmark 102 may be displayed as an overlay marking that point. The user may be prompted for input confirming that the landmark 102 marks a desirable reference point. Additional reference points are determined or input using techniques such as those described above. The external edge of the stomach is further detected using computer vision techniques, and an overlay 112 identifying that edge may be displayed. While not shown, a reference path may be determined and displayed as an overlay. Based on the reference path or points and the external edge shape and/or position, a guide path is determined, and an overlay of the guide path 104 is displayed. The user may adjust the guide path and/or offsets as described elsewhere in this application.

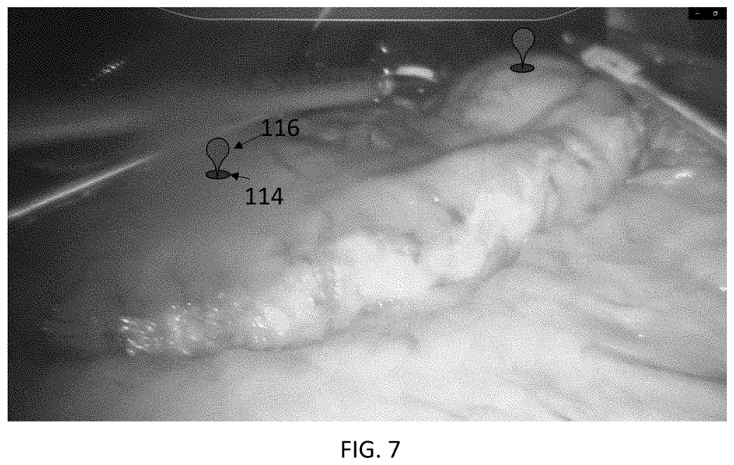

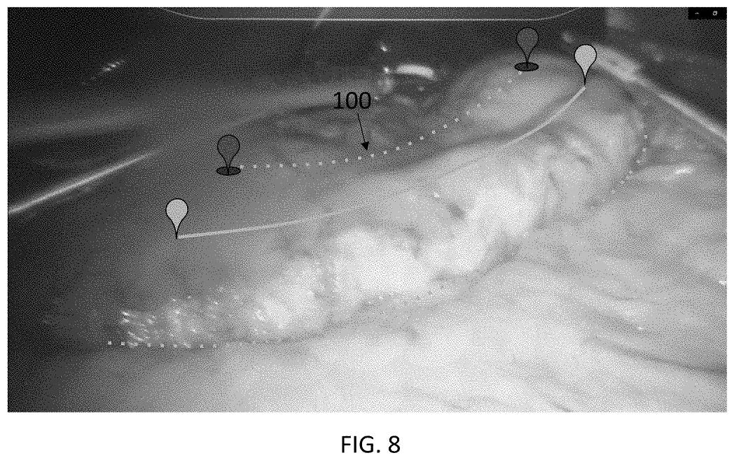

In a third embodiment shown in FIGS. 7-8, the system recognizes markings 114 physically placed on the stomach tissue, such as using ink, dye, sutures, etc. Overlays such as pins 116 or other icons may be generated and displayed on the endoscopic display marking the detected markings. The user may be prompted to give input confirming that the system should record those locations as reference points. The reference path is determined based on the reference points, and may be displayed as an overlay. The external edge of the stomach is further detected using computer vision techniques, and an overlay identifying that edge may be displayed. The guide path is defined between the reference path and the stomach's edge. The user may adjust the guide path using techniques described herein.

All patents and applications referenced herein, including for purposes of priority, are incorporated herein by reference.

What is claimed is:

1. A system for determining a guide path for display on an endoscopic display, comprising:
a camera positionable to capture image data corresponding to a treatment site;
at least one processor and at least one memory, the at least one memory storing instructions executable by said at least one processor to:
determine the positions of one or more reference points within the surgical environment,
based on the positions of the reference points, estimate or determine positions of guide points spaced from the reference point, and
generate output communicating the positions of the guide point(s).

2. The system of claim 1, wherein the instructions are further executable by the processor to generate an overlay marking the reference points and/or guide points on an image display displaying the image data.

3. The system of claim 1, wherein estimating or determining positions of guide points comprises determining a guide point spaced from a corresponding one of the reference points by a predetermined offset distance.

4. The system of claim 1, wherein estimating or determining positions of guide points comprises determining a first guide point spaced from a corresponding one of the reference points by a first predetermined offset distance and determining a second guide point spaced from a corresponding one of the reference points by a second predetermined offset distance.

5. A method for determining a guide path for display on an endoscopic display, comprising:
capturing image data corresponding to a treatment site;
using the image data, determining the positions of one or more reference points within the surgical environment,
based on the positions of the reference points, estimating or determining positions of guide points spaced from the reference point, and
displaying the image data on an image display;
displaying the positions of the guide point(s) as overlays on the image display.

6. The method of claim 5, wherein determining the positions of one or more reference points comprises receiving user input corresponding to the locations of said one or more reference points on an image display.

7. The method of claim 6, wherein determining the positions of one or more reference points comprises receiving user input digitally drawing said one or more reference points or paths as overlays on the image display.

8. The method of claim 5, wherein determining the positions of one or more reference points comprises using computer vision to detect anatomical landmarks, surgical devices, or physical markings at the surgical site.

9. The method of claim 8, wherein detecting a surgical device comprises using computer vision to determine a location of a bougie within a stomach captured in the image data, wherein at least one of the reference points is at the location.

10. The method of claim 7, wherein the user inputs the reference points or paths while observing a position of a bougie within a stomach captured in the image data, and wherein the guide points are a reference guide path for cutting a stapling a stomach.

11. The method of claim 5, wherein estimating or determining positions of guide points comprises determining a guide point spaced from a corresponding one of the reference points by a predetermined offset distance.

12. The method of claim 5, wherein estimating or determining positions of guide points comprises determining a first guide point spaced from a corresponding one of the reference points by a first predetermined offset distance and determining a second guide point spaced from a corresponding one of the reference points by a second predetermined offset distance.

13. The method of claim 11, further including receiving user input to modify the amount of the predetermined offset and determining a modified guide point spaced from the corresponding one of the reference points based on the modified offset.

14. The method of claim 13, wherein the user input comprised dragging an icon positioned at the guide point to the modified guide point.

15. The method of claim 13, wherein the method includes displaying a guide patch including the guide point, and wherein the user input comprises dragging a portion of the guide path to move the guide point to the modified guide point.

16. The method of claim 11, wherein the offset distance between the reference point and the guide point is a straight line distance.

17. The method of claim 11, wherein the offset distance between the reference point and the guide point is a geodesic distance following the topography of tissue surfaces between the reference and guide points.

18. The method of claim 11, wherein method includes generating an overlay displaying the offset distances.

19. The method of claim 11, wherein method includes generating an overlay displaying the path of the offset between the reference point and the guide point.
